(19) United States
(12) Patent Application Publication
(10) Pub. No.: US 2003/0232098 A1
(43) Pub. Date: Dec. 18, 2003

(54) Composition and method for treating tinnitus

(76) Inventor: Barry Keate, Salt Lake City, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 10/459,368

(22) Filed: Jun. 11, 2003

Related U.S. Application Data
(60) Provisional application No. 60/388,984, filed on Jun. 14, 2002.

(51) Int. Cl. A61K 35/78; A61K 31/315; A61K 31/30

(52) U.S. Cl. 424/752; 424/754; 514/494; 514/499

(57) ABSTRACT

Therapeutic compositions for the treatment of tinnitus are described which contain effective amounts of Ginkgo biloba, garlic and zinc. In formulations containing greater than 50 mg of zinc, the composition may further contain copper. Methods of treating patients having tinnitus include prescribing effective daily doses of the therapeutic compositions of the present invention over a sixty to ninety day period to maximize improvement in the condition, followed by a daily regimen of the therapeutic compositions.

### CROSS-REFERENCE TO RELATED APPLICATION

This is a non-provisional application claiming priority to provisional application Serial No. 60/388,984 filed Jun. 14, 2002.

### BACKGROUND OF THE INVENTION

1. Field of the Invention

This invention relates to therapeutic compositions and methods for treating disease conditions, and specifically relates to an improved compositions and methods for treating tinnitus.

2. Description of Related Art

Tinnitus is a condition of the body which is manifested by a ringing in one or both ears of a person. Tinnitus can be intermittent or a constant condition. It can be manifested as a single sound or tone or multiple tones. The perception of volume of the ringing or noise can range from subtle to very loud. The ringing experienced as a result of tinnitus can vary among individuals in intensity and timbre of ringing. Nonetheless, many persons who experience tinnitus are seriously affected by the constant ringing condition.

Over 50 million Americans experience tinnitus to some degree. Of these, about 12 million have tinnitus severe enough to seek medical attention. About two million patients are so seriously debilitated that they cannot function on a normal basis. They are unable to perceive normal sounds, such as conversation, and the condition is very disruptive to normal daily activities.

The physiological cause or causes of tinnitus are unknown. There are, however, several likely sources, all of which are known to trigger or worsen tinnitus. For example, exposure to loud noises can cause tinnitus. Seventy-five to eighty percent of all tinnitus patients have some level of noise-induced hearing loss. Certain medications can cause or increase tinnitus, and are known as ototoxic substances. Tinnitus can also be caused by ear or sinus infections. Cardiovascular disease is another typical cause of tinnitus, resulting in approximately three percent of all cases. Other cardiovascular conditions, such as heart murmurs, hypertension or hardening of the arteries can contribute to the condition.

A number of therapeutic compositions and treatment regimens have been proposed for tinnitus which fall into the area of conventional medical treatments. Examples of such compositions and treatments are disclosed in U.S. Pat. No. 6,524,619 to Pearson, et al. and U.S. Pat. No. 6,093,417 to Petrus. The compositions described in those patents include combinations of regulated substances which often require prescribed usage. Furthermore, the constituents of the compositions may cause other deleterious side effects in patients, thereby rendering them unsuitable for all patients as a curative.

There has been a movement in the last few decades toward exploring treatment methods and compositions that are outside the realm of conventional medicine. Specifically, there has been a move toward exploring natural methodologies for curing various medical conditions. To that end, compositions containing herbs have been proposed for treating tinnitus as a more natural medicament. Common herbs used in such compositions include garlic and Ginkgo biloba. The efficacy rate of such compositions is usually good.

It would be advantageous, however, to provide a composition for improved treatment of tinnitus which employs natural substances in an amount or concentration which increases the efficacy rate of treating the condition over known compositions.

### BRIEF SUMMARY OF THE INVENTION

In accordance with the present invention, compositions and methods are provided for treating tinnitus using naturally occurring substances in concentrations sufficient to treat and ameliorate or lessen the condition. The compositions of the present invention generally comprise an effective dose of Ginkgo biloba in combination with effective doses of garlic and zinc.

The use of herbs, including Ginkgo biloba and garlic, have been shown to produce a reduction in tinnitus symptoms in many patients suffering from the condition. Zinc has also been shown to provide therapeutic effects in treatment of tinnitus. However, while higher doses of zinc may produce increasing amelioration of the effects of tinnitus, higher doses of zinc are toxic for humans and cannot be prescribed.

Thus, the therapeutic composition of the present invention combines the efficacy of Ginkgo biloba and garlic with the efficacy of zinc in a synergistic combination that produces an increased treatment efficacy rate for tinnitus sufferers while enabling the use of lower, non-toxic doses of zinc.

In the present composition, the effective amount of Ginkgo biloba may range from between about 150 mg and about 720 mg per day, the effective daily dose of garlic in the composition may range from about 500 mg to about 2000 mg and the effective daily dose of zinc in the composition may range from between about 10 mg to about 100 mg. In doses which contain higher amounts of zinc, the present composition may also include copper in a ratio of between 1 mg to 2 mg of copper to 10 mg of zinc.

### DETAILED DESCRIPTION OF THE INVENTION

The compositions of the present invention generally comprise effective amounts of Ginkgo biloba and garlic in combination with effective, non-toxic doses of zinc as a treatment for tinnitus.

The effective daily dosage of Ginkgo biloba in the present therapeutic compositions may range from between about 150 mg and about 720 mg. Ginkgo biloba is produced in many forms, any of which may be suitably used in the present therapeutic compositions. However, a particularly suitable form is Ginkgo biloba leaf extract which contains a minimum of 24% Ginkgo flavone glycosides and 6% terpene lactones and a maximum of 5 ppm (parts per million) Ginkgolic acid, a strong allergen that can cause allergic reactions. That form of Ginkgo biloba may be obtained from any suitable source, including Japan Greenwave (Tokyo, Japan).

The effective daily dose of garlic in the compositions of the present invention may range from about 500 mg to about 2000 mg. Any type or formulation of dried or powdered garlic may be suitable for use in the invention. A particularly suitable form of garlic for use in the invention is odorless garlic, which is readily available from many sources, such as Ingredients International (City of Commerce, Calif.).

The effective daily dose of zinc in the composition may range from between about 10 mg to about 50 mg. The effective daily dose of zinc may be as great as 100 mg. In doses where zinc is present in a dosage of from about 50 mg to 100 mg, the present compositions may also include copper in a ratio of between 1 mg to 2 mg of copper to 10 mg of zinc. The inclusion of copper may be desirable to aid in absorption of zinc by the body.

Any type or formulation of zinc may be suitable for use in the present invention. In a preferred embodiment, however, the zinc is amino acid-chelated. Such amino acid-chelated zinc is available, for example, from Albion Laboratories of Clearfield, Utah.

The compositions of the present invention may be made by comminuting the dried forms of Ginkgo biloba, garlic and zinc to produce a homogeneous powdered mixture. The powdered mixture may then be placed in capsules or pressed into tablet form. Each capsule or tablet may typically contain twenty-five percent of the daily dosage of the therapeutic composition.

### EXAMPLES

Various therapeutic compositions were formulated in accordance with the present invention, as follows:

### Example I

150 mg of Ginkgo biloba leaf extract in dried form was combined with 500 mg of odorless garlic and 10 mg of zinc, both in powdered form. The constituents were comminuted until a homogeneous mixture was achieved. The combined mixture was then divided into four equal portions and placed in gelatin capsules to produce a daily dosage comprising four capsules.

### Example II

To 340 mg of Ginkgo biloba leaf extract was added 1000 mg of odorless garlic and 40 mg of zinc. The constituents were comminuted until a homogeneous mixture was achieved. The mixture was then divided into four equal portions and placed in gelatin capsules to produce a daily dosage comprising four capsules.

### Example III

An amount of 480 mg of Ginkgo biloba leaf extract was admixed with 1200 mg of odorless garlic and 20 mg of amino acid-chelated zinc. The admixture was comminuted together until a homogenous mixture was achieved. The resulting mixture was then formed into four tablets of equal portion to form a daily dosage of the therapeutic composition.

### Example IV

An amount of 720 mg of Ginkgo biloba leaf extract was admixed with 2000 mg odorless garlic, 100 mg of zinc and 15 mg copper. The mixture was comminuted until a homogenous mixture was achieved. The resulting mixture was then formed into four tablets of equal portion to form a daily dosage of the therapeutic composition.

### Test Results

A study was conducted on the effectiveness of zinc as a constituent of a therapeutic composition for treating tinnitus as compared with compositions containing only Ginkgo biloba and garlic. The study was conducted over a period of four months. Ten patients diagnosed with tinnitus were prescribed a daily dosage of 480 mg of Ginkgo biloba and 1200 mg odorless garlic, delivered in the form of four capsules taken orally per day, as a control group (Group A). Ten patients diagnosed with tinnitus were prescribed a daily dosage of 480 mg of Ginkgo biloba, 1200 mg of odorless garlic and 20 mg of chelated zinc, delivered in the form of four capsules taken orally per day (Group B). Ten patients diagnosed with tinnitus were prescribed a daily dosage of 480 mg of Ginkgo biloba, 1200 mg of odorless garlic and 40 mg of chelated zinc, delivered in the form of four capsules taken orally per day (Group C).

Standard questionnaires were used prior to testing to determine the degree of impairment of the patients and to prepare test results. The questionnaires used were the Tinnitus Handicap Inventory (THI) and Tinnitus Reaction Questionnaire (TRQ).

The patients in Group A reported a 14.7 point reduction in their tinnitus condition, or a 51% improvement. The patients in Group B reported a 19.3 point reduction in their tinnitus condition, or a 63% improvement. The patients in Group C reported a 21.5 point reduction in their tinnitus condition, or a 70% improvement. A statistically significant improvement (p<0.05) in the patients' conditions was shown through the TRQ evaluations.

With regard to subjective improvements reported by the patients in each test group, three of ten subjects in Group A reported at least a ten percent improvement in their conditions. Five of eight subjects in Group B reported at least a ten percent improvement in their condition and five of eight subjects in Group C reported at least a ten percent improvement in their tinnitus condition.

A method for treating tinnitus in accordance with the present invention comprises providing an effective daily dose of Ginkgo biloba, garlic and zinc. The effective daily dose may preferably be delivered in the form of four capsules or tablets taken daily, although the capsules or tablets may contain higher amounts of the composition ingredients such that fewer capsules or tablets deliver the same effective daily dosage. For example, with respect to the composition described in Example III above, each capsule or tablet may contain about 120 mg of Ginkgo biloba, 5 mg of zinc and 300 mg of odorless garlic. Tinnitus sufferers should typically continue the daily intake of the present compositions described herein for sixty to ninety days before maximized efficacy in reducing tinnitus symptoms is achieved.

The therapeutic compositions of the present invention are formulated for treatment of tinnitus in humans or other animals. The examples of compositions described herein are by way of example and not by way of limitation. Those skilled in the art will realize that certain modifications may be made to the compositions described herein to adapt the formulation for treatment of tinnitus.

What is claimed is:

1. A composition for treating tinnitus, comprising:
an effective daily dose of Ginkgo biloba in combination with an effective daily dose of garlic and an effective daily dose of zinc.

2. The composition of claim 1 wherein said zinc is amino acid-chelated form.

3. The composition of claim 2 wherein said garlic is odorless.

4. The composition of claim 3 wherein said Ginkgo biloba is in the form a leaf extract containing a minimum of 25% Gingko flavone glycosides and 6% terpene lactones and a maximum of 5 ppm Ginkgolic acid.

5. The composition of claim 1 wherein said effective daily dose of Ginkgo biloba ranges from about 150 mg to about 720 mg, said effective daily dose of garlic ranges from about 500 mg to about 2000 mg, and said effective daily dose of zinc ranges from about 10 mg to about 100 mg.

6. The composition of claim 5 wherein said zinc is present in an amount of 50 mg or greater, said composition further comprising copper in an amount of between 1 mg and 2 mg of copper per 10 mg of zinc.

7. The composition of claim 5 where said effective daily dose of Gingko biloba is 480 mg, said effective daily dose of garlic is 1200 mg and said effective daily dose of zinc is from between 20 mg and 40 mg.

8. The composition of claim 1 wherein said composition is formed as capsules for oral ingestion.

9. The composition of claim 1 wherein said composition is formed as tablets for oral ingestion.

10. A method for treating tinnitus, comprising:
providing a therapeutic composition containing an effective daily dosage of Ginkgo biloba in combination with an effective daily dosage of garlic and zinc;
administering said therapeutic composition to a patient experiencing tinnitus.

11. The method according to claim 10 wherein said composition of matter comprises an effective daily dose of Ginkgo biloba ranging from about 150 mg to about 720 mg, an effective daily dose of garlic ranging from about 500 mg to about 2000 mg, and an effective daily dose of zinc that ranges from about 10 mg to about 100 mg.

12. The method according to claim 10 wherein said therapeutic composition further comprises copper in an amount of between 1 mg and 2 mg per 10 mg of zinc when zinc is present in the composition in an amount equal to or greater than 50 mg.

13. The method according to claim 10 wherein said effective daily dosage of said composition of matter is administered in the form of four capsules or tablets taken daily.

14. The method according to claim 10 wherein said therapeutic composition is administered for at least sixty days to achieve maximum efficacy.
